(19) United States
(12) Patent Application Publication
(10) Pub. No.: US 2003/0133931 A1
(43) Pub. Date: Jul. 17, 2003

(54) Use of histamine H4 receptor antagonist for the treatment of inflammatory responses

(76) Inventors: Robin Thurmond, San Diego, CA (US); Siquan Sun, San Diego, CA (US); Lars Karlsson, La Jolla, CA (US)

Correspondence Address: Philip S. Johnson, Esq., Johnson & Johnson, One Johnson & Johnson Plaza, New Brunswick, NJ, 08933-7003, US

(21) Appl. No.: 10/036,648

(22) Filed: Dec. 21, 2001

(51) Int. Cl. A61K 39/395; C07K 16/28

(52) U.S. Cl. 424/143.1; 530/388.22

(57) ABSTRACT

The present invention relates to the use of histamine H4 receptor modulators for the prevention, treatment, induction, or other desired modulation of inflammatory responses, inflammation, or diseases and/or conditions that are modulated, affected or caused by inflammation or inflammatory responses. The present invention also relates to the use of histamine H4 receptor modulators for the prevention, treatment, induction, or other desired modulation of polymorphonuclear leukocyte responses, such as migration to a particular site, or diseases and/or conditions that are modulated, affected or caused by polymorphonuclear leukocytes. The present invention also relates to the use of histamine H4 receptor modulators for the prevention, treatment, induction, or other desired modulation of mast cell responses, such as de-granulation, or diseases and/or conditions that are modulated, affected or caused by mast cells.

### FIELD OF THE INVENTION

The present invention relates to the use of histamine H4 receptor modulators for the prevention, treatment, induction, or other desired modulation of inflammatory responses, inflammation, or diseases and/or conditions that are modulated, affected or caused by inflammation or inflammatory responses.

### BACKGROUND OF THE INVENTION

Histamine is a multifunctional chemical transmitter that signals through cell surface receptors that are linked to intracellular pathways via guanine nucleotide binding proteins. This class of histamine binding cell surface receptor is part of a broad family of receptors called G-protein coupled receptors or GPCRs. There are currently four subtypes of histamine receptors that have been defined pharmacologically and have been divided into H1, H2, H3, and H4 classifications (Hill, et al. 1997). The H1 histamine receptor has been cloned (Yamashita et al. 1991) and is the target of drugs such as diphenhydramine to block the effects of histamine on smooth muscle in allergic responses. The H2 histamine receptor has been cloned (Gantz et al. 1991) and is the target of drugs such as ranitidine to block the effects of histamine on acid secretion in the stomach. The H3 histamine receptor, which was hypothesized to exist in 1983 (Arrang et al. 1983), has been cloned (Lovenberg et al., 1999) and is currently a target for development of central nervous system drugs. There are numerous additional functions of histamine in humans which may be mediated by histamine receptors of unknown class. For example, histamine is a chemotactic factor for leukocytes, causing their accumulation in areas of allergic challenge such as skin, nose, eyes and lungs (de Vos, 1999).

### SUMMARY OF THE INVENTION

The present invention relates to the use of histamine H4 receptor antagonists for the treatment and/or prevention of inflammation and/or inflammatory responses, and the diseases and conditions mediated by inflammation and/or inflammatory responses. Modulators of the histamine H4 receptor may be used for modulating inflammatory responses in mammals, including the induction as well as the inhibition of the inflammatory responses depending on whether the histamine H4 receptor modulator is an H4 receptor activity agonist, inverse agonist, or antagonist. Inflammation and inflammatory responses mediated by leukocytes or mast cells are inhibited by treatment with antagonists or inhibitors of the histamine H4 receptor.

### BRIEF DESCRIPTION OF THE DRAWINGS

FIG. 1: The amount of polymorphonuclear cells in the peritoneal lavage after local application of zymosan. The circles represent the number for each individual mouse and the bar is the average.

FIG. 2: The amount of polymorphonuclear cells in the peritoneal lavage after local application of monosodium urate crystals. The circles represent the number for each individual mouse and the bar is the average.

FIG. 3: The difference in ear thickness between the ear that was exposed to croton oil and the unexposed ear. The circles represent the number for each individual mouse and the bar is the average.

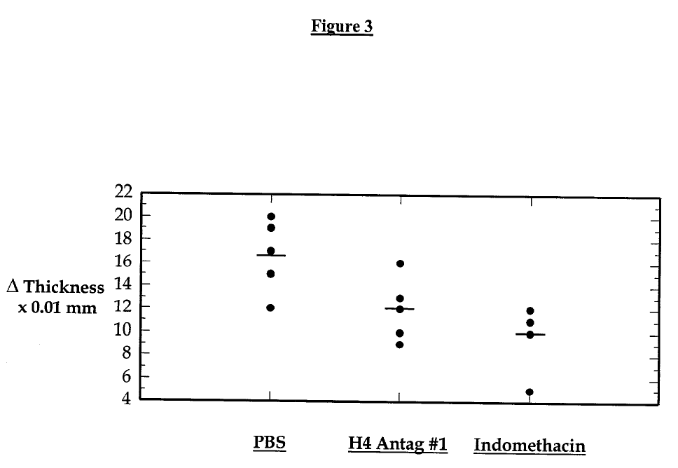

### DETAILED DESCRIPTION OF THE INVENTION

DNA molecules encoding a mammalian histamine H4 receptor have been cloned and characterized and represent a novel member of the class of receptors that couple to G-proteins [Changlu Liu, Sandy J. Wilson, Chester Kuei, and Timothy W. Lovenberg, J. Pharmacol. Experimental Therapeutics, (2001) 299(1):121-130]. Using a recombinant expression system, functional DNA molecules encoding these histamine H4 receptors have been isolated from mouse, rat, guinea pig, and human. The biological and structural properties of these proteins are disclosed, as is the amino acid and nucleotide sequence. The recombinant protein is useful for a variety of purposes, including but not limited to identifying modulators of the human histamine H4 receptor. The histamine H4 receptors of mouse, rat, and guinea pig have a variety of uses, including, but not limited to, resolving pharmacological differences observed between different mammalian species, particularly since guinea pig, rat, and murine species are commonly used in pre-clinical evaluation of new chemical entities. Such modulators can include agonists, antagonists, and inverse agonists. Modulators identified in the assays disclosed herein are useful, for example, as therapeutic agents, prophylactic agents, and diagnostic agents. Indications for said therapeutic agents include, but are not limited to, asthma, allergy, inflammation, cardiovascular and cerebrovascular disorders, non-insulin dependent diabetes mellitus, hyperglycemia, constipation, arrhythmia, disorders of the neuroendocrine system, stress, and spasticity, as well as acid secretion, ulcers, airway constriction, and prostate dysfunction. The recombinant DNA molecules, and portions thereof, have a variety of uses including but not limited to isolating homologues of the DNA molecules, identifying and isolating genomic equivalents of the DNA molecules, and identifying, detecting or isolating mutant forms of the DNA molecules. The human histamine H4 receptor, as used herein, refers to protein that can specifically function as a receptor for histamine of the H4 subclass.

Inflammation is a normal protective or defensive response of mammals elicited by events such as trauma or other physical stimulation, chemical stimulation, infection or presence of a biological agent, or invasion by a foreign body. The inflammatory response is characterized by pain, increased temperature, redness, swelling and, in some cases, inhibition or loss of function. All or only some of these signs may be present at a given time, but no one of them is necessarily always present. These symptoms are due to a series of interrelated events that result from the action of cells, as well as chemicals or substances produced by cells, and can include vascular dilation, the exudation of fluids and plasma proteins, and the migration of leukocytes into the injured or stimulated area. There is a fine balance between the necessary inflammatory response that is required to ward off infections or other stimuli and an over-response that can lead to inflammatory diseases. Many pathologic conditions, such as allergies, asthma, chronic obstructed pulmonary disease (COPD), arthrosclerosis, and autoimmune diseases, including rheumatoid arthritis and lupus, are characterized by excessive or prolonged inflammation. Most of these conditions are driven by the recruitment of leukocytes to the area of inflammation and therefore agents that block this can have a major therapeutic effect.

Inflammation mediated diseases or conditions that are well known in the art include, but are not limited to, active inflammation, acute inflammation, adhesive inflammation, allergic inflammation, alterative inflammation, atrophic inflammation, catarrhal inflammation, chronic inflammation, chronic active inflammation, degenerative inflammation, exudative inflammation, fibrinopurluent inflammation, fibrinous inflammation, fibroid inflammation, granulomatous inflammation, hyperplastic inflammation, immune inflammation, interstitial inflammation, necrotic inflammation, productive inflammation, proliferative inflammation, pseudomembranous inflammation, purluent inflammation, sclerosing inflammation, serofibrinous inflammation, serous inflammation, sub-acute inflammation, and supperative inflammation.

Mast cells are an important part of the inflammatory response, and mast cell de-granulation (exocytosis) leads to an inflammatory response that may be initially characterized by a wheal and flare reaction that is modulated by histamine. A wide variety of stimuli may cause the activation of mast cells, and subsequently cause them to migrate to a particular location (recruitment) and/or to undergo de-granulation. These stimuli may be immunologic (such as antibodies or allergens) or non-immunologic (such as chemical agents) in nature. Mast cell activation initiates allergic inflammatory responses, which in turn causes the recruitment of other effector cells that further contribute to the inflammatory response. The histamine H1 receptor is crucial for this type of inflammatory response, while the histamine H2 receptors modulate gastric acid secretion and the histamine H3 receptors affect neurotransmitter release in the central nervous system. Recently, the histamine receptor H4 has been cloned and demonstrated to be expressed in a variety of cells, including but not limited to, leukocytes and mast cells.

Numerous medical texts are published, are well known, and are readily available to those of skill in the relevant art fields. In addition, numerous scientific and medical research publications have been published in the field of inflammation. Examples of widely available published textbooks on the subject of inflammation include J. I. Gallin and R. Snyderman, Inflammation: Basic Principles and Clinical Correlates, 3^{rd} Edition, (Lippincott Williams & Wilkins, Philadelphia, 1999); V. Stvrtinova, J. Jakubovsky and I. Hulin, “Inflammation and Fever”, Pathophysiology Principles of Diseases (Textbook for Medical Students, Academic Press, 1995); Cecil et al., Textbook Of Medicine, 18^{th} Edition (W. B. Saunders Company, 1988); and Steadmans Medical Dictionary.

The present invention demonstrates that the histamine H4 receptor is involved the inflammatory response, and particularly involved in leukocyte recruitment to the site of inflammation and that antagonists for this receptor are anti-inflammatory. The present invention provides methods for modulating inflammatory responses that are directly or indirectly mediated by the histamine H4 receptor. The present invention also provides methods for inhibiting, preventing, ameliorating, inducing, or otherwise affecting inflammatory responses that are mediated by the histamine H4 receptor, through the treatment of a mammal with modulators of the histamine H4 receptor. Modulators of the histamine H4 receptor that are useful in the method of the present invention include, but are not limited to, antibodies and antibody fragments that bind the histamine H4 receptor, inhibitors, activators, antagonists, agonists and reverse agonists of the histamine H4 receptor, including, but not limited to, proteins, nucleic acids, or other organic molecules. These modulators are useful for administration to humans in need thereof, and are also useful for veterinary purposes to administer to non-human animals, including but not limited to non-human mammals.

Histamine is a biogenic amine transmitter that functions in some capacity in nearly all physiological and pathophysiological situations. Histamine acts as a neurotransmitter and neuromodulator in the central nervous system, mediates inflammatory and allergic responses, regulates airway function, controls acid secretion in the stomach, regulates cardiovascular function as well as arterial and venous responses and is without doubt involved in processes yet to be determined. The histamine receptors that mediate these effects are not completely characterized. One way to understand which histamine receptors are involved in these processes is to develop chemical modulators (such as agonists, antagonists, and inverse agonists) of the receptors as research tools and therapeutic entities. Recombinant host cells expressing the mammalian histamine H4 receptor can be used to provide materials for a screening method to identify such agonists and antagonists. As such, this invention of the mammalian histamine H4 receptor directly teaches a way to identify new agonists and antagonists that may prove useful as research tools or may be used as therapeutics to treat disorders directly or indirectly involving histamine receptors, such as inflammatory responses and inflammation. Assays to detect compound interaction or modulation of the histamine H4 receptor include, but are not limited to, direct ligand binding assays, competitive (or displacement) ligand binding assays, or functional assays that measure the response of the receptor to the ligand, for example by production of cAMP. Although these assays are well known to those skilled in the art, they were previously not possible prior to obtaining the recombinant molecules taught herein.

Monospecific antibodies to mammalian histamine H4 receptor are purified from mammalian antisera containing antibodies reactive against mammalian histamine H4 receptor or are prepared as monoclonal antibodies reactive with mammalian histamine H4 receptor using the technique of Kohler and Milstein, Nature (1975) 256:495-497. Monospecific antibody as used herein is defined as a single antibody species or multiple antibody species with homogenous binding characteristics for mammalian histamine H4 receptor. Homogenous binding as used herein refers to the ability of the antibody species to bind to a specific antigen or epitope, such as those associated with the mammalian histamine H4 receptor, as described above. Mammalian histamine H4 receptor specific antibodies are raised by immunizing animals such as mice, rats, guinea pigs, rabbits, goats, horses and the like, with rabbits being preferred, with an appropriate concentration of mammalian histamine H4 receptor either with or without an immune adjuvant.

Preimmune serum is collected prior to the first immunization. Each animal receives between about 0.1 mg and about 1000 mg of mammalian histamine H4 receptor associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of mammalian histamine H4 receptor in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of the antigen in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20° C.

Monoclonal antibodies (mAb) reactive with mammalian histamine H4 receptor are prepared by immunizing inbred mice, preferably Balb/c, with mammalian histamine H4 receptor and any fragments thereof. The mice are immunized by the IP or SC route with about 0.1 mg to about 10 mg, preferably about 1 mg, of mammalian histamine H4 receptor in about 0.5 ml buffer or saline incorporated in an equal volume of an acceptable adjuvant, as discussed above. Freund's complete adjuvant is preferred. The mice receive an initial immunization on Day 0 and are rested for about three to thirty weeks. Immunized mice are given one or more booster immunizations of about 0.1 to about 10 mg of mammalian histamine H4 receptor in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to, mouse myelomas P3/NS1/Ag 4-1; MPC-11; S-194 and Sp 2/0, with Sp 2/0 being generally preferred. The antibody producing cells and myeloma cells are fused in polyethylene glycol, about 1000 mol. wt., at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells on about Days 14, 18, and 21 and are screened for antibody production by an immunoassay such as solid phase immunoradioassay (SPIRA) using mammalian histamine H4 receptor as the antigen. The culture fluids are also tested in the Ouchterlony precipitation assay to determine the isotype of the mAb. Hybridoma cells from antibody positive wells are cloned by a technique, such as the soft agar technique of MacPherson, “Soft Agar Techniques”, Tissue Culture Methods and Applications (Kruse and Paterson (Eds.), Academic Press, 1973).

Monoclonal antibodies are produced in vivo by injection of pristane primed Balb/c mice, approximately 0.5 ml per mouse, with about 2×10⁶ to about 6×10⁶ hybridoma cells about four days after priming. Ascites fluid is collected at approximately eight to twelve days after cell transfer and the monoclonal antibodies are purified by techniques known in the art.

In vitro production of anti-mammalian histamine H4 receptor mAb is carried out by growing the hybridoma in DMEM containing about 2% fetal calf serum to obtain sufficient quantities of the specific mAb. The mAb are purified by techniques known in the art. Antibody titers of ascites or hybridoma culture fluids are determined by various serological or immunological assays which include, but are not limited to, precipitation, passive agglutination, enzyme-linked immunosorbent antibody (ELISA) technique and radioimmunoassay (RIA) techniques. Similar assays are used to detect the presence of mammalian histamine H4 receptor in body fluids or tissue and cell extracts.

It is readily apparent to those skilled in the art that the above described methods for producing monospecific antibodies may be utilized to produce antibodies specific for mammalian histamine H4 receptor polypeptide fragments, or full-length nascent mammalian histamine H4 receptor polypeptide, or the individual mammalian histamine H4 receptor epitopes. Specifically, it is readily apparent to those skilled in the art that monospecific antibodies may be generated that are specific for only one species of mammalian histamine H4 receptor portion or the fully functional histamine H4 receptor. It is also readily apparent to one of ordinary skill in the art that antibodies that are specific for the histamine H4 receptor may cause a change in the functional activity of the receptor, including but not limited to, causing the receptor to be activated or inactivated, blocked from binding its ligand, blocked from releasing its bound ligand, or prevented from functioning in the normal fashion associated with a histamine H4 receptor.

Nucleotide sequences that are complementary to the human histamine H4 receptor encoding DNA sequence can be synthesized for antisense therapy. These antisense molecules may be DNA, stable derivatives of DNA such as phosphorothioates or methylphosphonates, RNA, stable derivatives of RNA such as 2′-O-alkylRNA, or other human histamine H4 receptor antisense oligonucleotide mimetics. Human histamine H4 receptor antisense molecules may be introduced into cells by microinjection, liposome encapsulation or by expression from vectors harboring the antisense sequence. Human histamine H4 receptor antisense therapy may be particularly useful for the treatment of diseases where it is beneficial to reduce human histamine H4 receptor activity.

Human histamine H4 receptor gene therapy may be used to introduce human histamine H4 receptor into the cells of target organisms. The human histamine H4 receptor gene can be ligated into viral vectors which mediate transfer of the human histamine H4 receptor DNA by infection of recipient host cells. Suitable viral vectors include retrovirus, adenovirus, adeno-associated virus, herpes virus, vaccinia virus, poliovirus and the like. Alternatively, human histamine H4 receptor DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo human histamine H4 receptor gene therapy. Human histamine H4 receptor gene therapy may be particularly useful for the treatment of diseases where it is beneficial to elevate human histamine H4 receptor activity.

The present invention is also directed to methods for screening for compounds that modulate the expression of DNA or RNA encoding mammalian histamine H4 receptor as well as the function of mammalian histamine H4 receptor protein in vitro and in vivo. Compounds that modulate these activities may be DNA, RNA, peptides, proteins, or non-proteinaceous organic molecules. Compounds may modulate by increasing or attenuating the expression of DNA or RNA encoding mammalian histamine H4 receptor, or the function of mammalian histamine H4 receptor protein. Compounds that modulate the expression of DNA or RNA encoding mammalian histamine H4 receptor or the function of mammalian histamine H4 receptor protein may be detected by a variety of assays. The assays may be a simple “yes/no” assay to determine whether there is a change in expression of nucleic acid encoding the receptor, or a change in the function or activity of the receptor protein. The assay may be made quantitative by comparing the expression or function of a test sample with the levels of receptor expression or receptor protein function in a standard sample. Modulators identified in this process are useful as therapeutic agents, research tools, and diagnostic agents.

Kits containing mammalian histamine H4 receptor DNA or RNA, antibodies to mammalian histamine H4 receptor, or mammalian histamine H4 receptor protein may be prepared. Such kits are used to detect DNA that hybridizes to mammalian histamine H4 receptor DNA or to detect the presence of mammalian histamine H4 receptor protein or peptide fragments in a sample. Such characterization is useful for a variety of purposes including but not limited to forensic analyses, diagnostic applications, and epidemiological studies.

The DNA molecules, RNA molecules, recombinant protein and antibodies of the present invention may be used to screen and measure levels of mammalian histamine H4 receptor DNA, mammalian histamine H4 receptor RNA or mammalian histamine H4 receptor protein. The recombinant proteins, DNA molecules, RNA molecules and antibodies lend themselves to the formulation of kits suitable for the detection and typing of mammalian histamine H4 receptor. Such a kit would comprise a compartmentalized carrier suitable to hold in close confinement at least one container. The carrier would further comprise reagents such as recombinant mammalian histamine H4 receptor protein or anti-mammalian histamine H4 receptor antibodies suitable for detecting mammalian histamine H4 receptor. The carrier may also contain a means for detection such as labeled antigen or enzyme substrates or the like.

Nucleotide sequences that are complementary to the mammalian histamine H4 receptor encoding DNA sequence can be synthesized for antisense therapy. These antisense molecules may be DNA, stable derivatives of DNA such as phosphorothioates or methylphosphonates, RNA, stable derivatives of RNA such as 2′-O-alkylRNA, or other mammalian histamine H4 receptor antisense oligonucleotide mimetics. Mammalian histamine H4 receptor antisense molecules may be introduced into cells by microinjection, liposome encapsulation or by expression from vectors harboring the antisense sequence. mammalian histamine H4 receptor antisense therapy may be particularly useful for the treatment of diseases where it is beneficial to reduce mammalian histamine H4 receptor activity.

Mammalian histamine H4 receptor gene therapy may be used to introduce mammalian histamine H4 receptor into the cells of target organisms. The mammalian histamine H4 receptor gene can be ligated into viral vectors that mediate transfer of the mammalian histamine H4 receptor DNA by infection of recipient host cells. Suitable viral vectors include retrovirus, adenovirus, adeno-associated virus, herpes virus, vaccinia virus, poliovirus and the like. Alternatively, mammalian histamine H4 receptor DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo mammalian histamine H4 receptor gene therapy. Mammalian histamine H4 receptor gene therapy may be particularly useful for the treatment of diseases where it is beneficial to elevate mammalian histamine H4 receptor activity.

Pharmaceutically useful compositions comprising mammalian histamine H4 receptor DNA, mammalian histamine H4 receptor RNA, or mammalian histamine H4 receptor protein, or modulators of mammalian histamine H4 receptor activity, may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, or modulator.

Therapeutic or diagnostic compositions of the invention are administered to an individual in amounts sufficient to treat or diagnose disorders in which modulation of mammalian histamine H4 receptor-related activity is indicated. The effective amount may vary according to a variety of factors such as the individual's condition, weight, sex and age. Other factors include the mode of administration. The pharmaceutical compositions may be provided to the individual by a variety of routes such as subcutaneous, topical, oral and intramuscular.

The term “chemical derivative” describes a molecule that contains additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Compounds identified according to the methods disclosed herein may be used alone at appropriate dosages defined by routine testing in order to obtain optimal inhibition of the mammalian histamine H4 receptor or its activity while minimizing any potential toxicity. In addition, co-administration or sequential administration of other agents may be desirable.

The present invention also has the objective of providing suitable topical, oral, systemic and parenteral pharmaceutical formulations for use in the novel methods of treatment of the present invention. The compositions containing compounds or modulators identified according to this invention as the active ingredient for use in the modulation of mammalian histamine H4 receptor receptors can be administered in a wide variety of therapeutic dosage forms in conventional vehicles for administration. For example, the compounds or modulators can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the compound desired can be employed as a mammalian histamine H4 receptor modulating agent.

The daily dosage of the products may be varied over a wide range from 0.01 to 1,000 mg per patient, per day. For oral administration, the compositions are preferably provided in the form of scored or un-scored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. The dosages of the mammalian histamine H4 receptor modulators are adjusted when combined to achieve desired effects. On the other hand, dosages of these various agents may be independently optimized and combined to achieve a synergistic result wherein the pathology is reduced more than it would be if either agent were used alone.

Advantageously, compounds or modulators of the present invention may be administered in a single daily dose, or the total daily dosage may be administered in divided doses of two, three or four times daily. Furthermore, compounds or modulators for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds or modulators of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

In the methods of the present invention, the compounds or modulators herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars, such as glucose or beta-lactose, corn sweeteners, natural and synthetic gums, such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methylcellulose, agar, bentonite, xanthan gum and the like.

For liquid forms the active drug component can be combined in suitably flavored suspending or dispersing agents such as the synthetic and natural gums, for example, tragacanth, acacia, methyl-cellulose and the like. Other dispersing agents that may be employed include glycerin and the like. For parenteral administration, sterile suspensions and solutions are desired. Isotonic preparations that generally contain suitable preservatives are employed when intravenous administration is desired.

Topical preparations containing the active drug component can be admixed with a variety of carrier materials well known in the art, such as, e.g., alcohols, aloe vera gel, allantoin, glycerine, vitamin A and E oils, mineral oil, PPG2 myristyl propionate, and the like, to form, e.g., alcoholic solutions, topical cleansers, cleansing creams, skin gels, skin lotions, and shampoos in cream or gel formulations.

The compounds or modulators of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds or modulators of the present invention may also be coupled with soluble polymers as targetable drug carriers. Such polymers can include polyvinyl-pyrrolidone, pyran copolymer, polyhydroxypropylmethacryl-amidephenol, polyhydroxy-ethylaspartamidephenol, or polyethyl-eneoxidepolylysine substituted with palmitoyl residues. Furthermore, the compounds or modulators of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For oral administration, the compounds or modulators may be administered in capsule, tablet, or bolus form or alternatively they can be mixed in the animals feed. The capsules, tablets, and boluses are comprised of the active ingredient in combination with an appropriate carrier vehicle such as starch, talc, magnesium stearate, or di-calcium phosphate. These unit dosage forms are prepared by intimately mixing the active ingredient with suitable finely-powdered inert ingredients including diluents, fillers, disintegrating agents, and/or binders such that a uniform mixture is obtained. An inert ingredient is one that will not react with the compounds or modulators and which is non-toxic to the animal being treated. Suitable inert ingredients include starch, lactose, talc, magnesium stearate, vegetable gums and oils, and the like. These formulations may contain a widely variable amount of the active and inactive ingredients depending on numerous factors such as the size and type of the animal species to be treated and the type and severity of the infection. The active ingredient may also be administered as an additive to the feed by simply mixing the compound with the feedstuff or by applying the compound to the surface of the feed. Alternatively the active ingredient may be mixed with an inert carrier and the resulting composition may then either be mixed with the feed or fed directly to the animal. Suitable inert carriers include corn meal, citrus meal, fermentation residues, soya grits, dried grains and the like. The active ingredients are intimately mixed with these inert carriers by grinding, stirring, milling, or tumbling such that the final composition contains from 0.001 to 5% by weight of the active ingredient.

The compounds or modulators may alternatively be administered parenterally via injection of a formulation consisting of the active ingredient dissolved in an inert liquid carrier. Injection may be either intramuscular, intra-ruminal, intratracheal, or subcutaneous. The injectable formulation consists of the active ingredient mixed with an appropriate inert liquid carrier. Acceptable liquid carriers include the vegetable oils, such as peanut oil, cottonseed oil, sesame oil and the like as well as organic solvents such as solketal, glycerol formal and the like. As an alternative, aqueous parenteral formulations may also be used. The vegetable oils are the preferred liquid carriers. The formulations are prepared by dissolving or suspending the active ingredient in the liquid carrier such that the final formulation contains from 0.005 to 10% by weight of the active ingredient.

Topical application of the compounds or modulators is possible through the use of a liquid drench or a shampoo containing the instant compounds or modulators as an aqueous solution or suspension. These formulations generally contain a suspending agent such as bentonite and normally will also contain an antifoaming agent. Formulations containing from 0.005 to 10% by weight of the active ingredient are acceptable. Preferred formulations are those containing from 0.01 to 5% by weight of the instant compounds or modulators.

The following examples are provided for the purpose of illustrating the present invention without, however, limiting the same thereto.

### EXAMPLE 1

### The Inhibition of Zymosan Induced Peritonitis in Mice by Histamine H4 Receptor Antagonists

This example demonstrates the discovery for the first time that histamine H4 receptor antagonists can block the peritonitis induced by zymosan, which is the insoluble polysaccharide component on the cell wall of Saccharomyces cerevisiae. This is commonly used to induce peritonitis in mice and appears to act in a mast cell dependent manner.

Materials and Methods

Animals

Male out-bred Swiss albino mice were purchased from Bantin and Kingman (T.O. strain; Hull, Humberside) and maintained on a standard chow pellet diet with tap water ad libitum and a twelve-hour light/dark cycle. All animals were housed for at least three days prior to experimentation to allow body weight to reach ˜30 grams on the day of the experiment. For this particular experiment, body weight was 30.5±0.3 (n=32). Animals were briefly (30 to 60 seconds) anesthetized with halothane for all s.c. and i.p. treatments described below.

Drug Treatment and Experimental Design

Drugs were stored at room temperature, in the dark. On the day of the experiment, drugs were dissolved in sterile PBS as depicted below, and generously vortexed.

H4 ANTAG #1 was prepared at 10 mg/5 ml, and injected at 5 ml/kg.

Imetit was prepared at 5 mg/5 ml, and injected at 5 ml/kg.

Thioperamide was prepared at 5 mg/5 ml, and injected at 5 ml/kg.

Time

Time −15 minutes: Compounds or PBS administered s.c. at the reported doses.

Time 0: At Time 0, mice received 1 mg zymosan A (Sigma) i.p.

Time +2 hours: Compounds or PBS administered s.c. at the reported doses.

Time +4 hours: Peritoneal cavities were washed four hours later with 3 ml of PBS containing 3 mM EDTA, and the number of migrated leukocytes determined, by taking an aliquot (100 μl) of the lavage fluid and diluting 1:10 in Turk's solution (0.01% crystal violet in 3% acetic acid). The samples were then vortexed and 10 μl of the stained cell solution were placed in a Neubauer haemocytometer. Differential cell counts were performed using a light microscope (Olympus B061). In view of their chromatic characteristics and their nucleus and cytoplasm appearance, polymorphonuclear leukocytes (PMN; >95% neutrophils) could be easily identified.

Experimental groups are described below:

PBS+zymosan n=8

H4 ANTAG #1+zymosan n=8

Imetit+zymosan n=8

Thiopermide+zymosan n=8

Statistics

Data are shown for single mice, and also shown as mean±S.d. or SEM of eight mice per group. The percent of inhibition is also shown. Statistical differences were determined by Anova followed by Bonferroni's post-hoc test.

Results

TABLE 1
Effect of H4 Antagonist Compounds on Zymosan Peritonitis

| | | PMN | | | | P value |
|---|---|---|---|---|---|---|
| Treatment | n | (10⁶ per mouse) | Mean | Sd | Sem | (% inhib) |
| PBS | 1 | 15.9 | 17.2 | 2.4 | 0.8 | |
| (s.c.) | 2 | 18.3 | | | | |
| | 3 | 16.2 | | | | |
| | 4 | 17.4 | | | | |
| | 5 | 19.8 | | | | |
| | 6 | 12.6 | | | | |
| | 7 | 19.8 | | | | |
| | 8 | 17.7 | | | | |
| H4 ANTAG #1 | 1 | 9.9 | 6.6 | 2.7 | 1.0 | 0.001 |
| (10 mg/kg; s.c.) | 2 | 3.6 | | | | (−62%) |
| | 3 | 9.3 | | | | |
| | 4 | 3.3 | | | | |
| | 5 | 8.1 | | | | |
| | 6 | 5.1 | | | | |
| | 7 | 6.9 | | | | |
| Imetit | 1 | 19.8 | 17.3 | 2.6 | 0.9 | n.s. |
| (5 mg/kg; s.c.) | 2 | 17.1 | | | | — |
| | 3 | 14.1 | | | | |
| | 4 | 15.3 | | | | |
| | 5 | 21.3 | | | | |
| | 6 | 17.7 | | | | |
| | 7 | 14.1 | | | | |
| | 8 | 18.6 | | | | |
| Thioperamide | 1 | 9.3 | 9.3 | 3.4 | 1.2 | 0.001 |
| (5 mg/kg; s.c.) | 2 | 16.5 | | | | (−46%) |
| | 3 | 7.2 | | | | |
| | 4 | 10.8 | | | | |
| | 5 | 5.4 | | | | |
| | 6 | 9.9 | | | | |
| | 7 | 6.9 | | | | |
| | 8 | 8.1 | | | | |

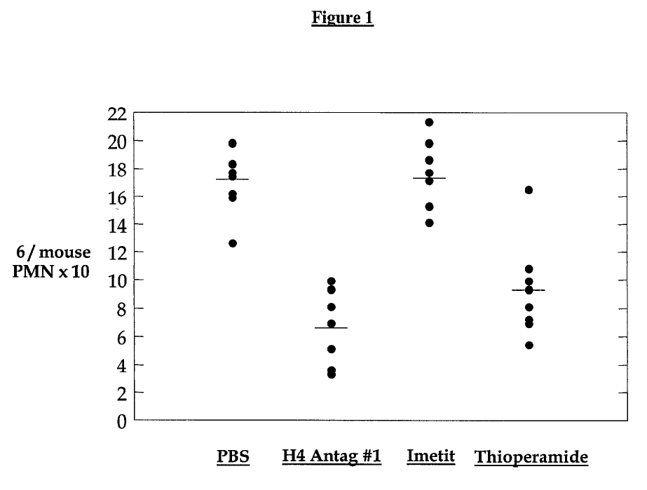

From data analysis it can be seen that zymosan produced a leukocyte extravasation response that was intense at the four-hour time point. Treatment with 10 mg/kg H4 ANTAG #1 significantly reduced PMN influx (compare PBS group to H4 ANTAG #1 group in Table 1 and FIG. 1). The degree of inhibition was >60%. Imetit (5 mg/kg) was inactive, whereas a significant inhibitory effect was attained by 5 mg/kg thioperamide.

Conclusion

To conclude, this study demonstrates that a histamine H4 receptor antagonist, H4 ANTAG #1, given at the dose of 10 mg/kg, is effective in reducing PMN accumulation in an experimental model of cell recruitment in response to local application of zymosan in the mouse peritoneal cavity. Furthermore, thioperamide which is a dual H3/H4 receptor antagonist, is also effective. The dual H3/H4 receptor agonist, Imetit, does not have any effect. This shows that an antagonist of the histamine H4 receptor can block inflammation induced by zymosan.

### EXAMPLE 2

### The Inhibition of Sodium Urate Crystal Induced Peritonitis in Mice by Histamine H4 Receptor Antagonists

This example demonstrates the discovery for the first time that histamine H4 receptor antagonists can block the peritonitis induced by sodium urate crystals. Such crystals are the primary cause of the inflammation associated with acute gouty arthritis.

Materials and Methods

Animals

Male out-bred Swiss albino mice were purchased from Bantin and Kingman (T.O. strain; Hull, Humberside) and maintained on a standard chow pellet diet with tap water ad libitum and a twelve-hour light/dark cycle. All animals were housed for at least three days prior to experimentation to allow body weight to reach ˜30 g on the day of the experiment. For this particular experiment, body weight was 30±1 (n=32).

Drug Treatment and Experimental Design

H4 ANTAG #1 was stored at room temperature in the dark. On the day of the experiment, H4 ANTAG #1 was dissolved in phosphate buffered saline (PBS) to a concentration of 3 mg/ml. At Time −15 minutes, H4 ANTAG #1 was administered s.c. at the dose of 10 mg/kg, whereas the control group received the vehicle alone (10 ml/kg). Mice received 3 mg mono sodium urate crystals (MSU) given intra-peritoneally at Time 0. At Time +2 hours and Time +4 hours, H4 ANTAG #1 (10 mg/kg) or vehicle (10 ml/kg) were given s.c.

Time +6 hours: Peritoneal cavities were washed 6 hours later with 3 ml of PBS containing 3 mM EDTA, and the number of migrated leukocytes determined, by taking an aliquot (100 μl) of the lavage fluid and diluting 1:10 in Turk's solution (0.01% crystal violet in 3% acetic acid). The samples were then vortexed and 10 μl of the stained cell solution were placed in a Neubauer hematocytometer. Differential cell counts were performed using a light microscope (Olympus B061). In view of their chromatic characteristics and their nucleus and cytoplasm appearance, cells polymorphonuclear cells (PMN, >95% neutrophils) could be easily differentiated.

Experimental groups are described below:

Vehicle+MSU crystals n=8

H4 ANTAG #1+MSU crystals n=8

Statistics

Data are shown for single mice, and also shown as mean±S.d. of (n) mice per group. Statistical differences were determined by Student's t test. A P value <0.05 was taken as significant.

Results

TABLE 2
Effect of H4 ANTAG #1 on MSU-Induced Leukocyte Migration
as Evaluated at the 6-Hour Time-Point

| | | PMN | | | | P value |
|---|---|---|---|---|---|---|
| Treatment | n | (10⁶ per mouse) | Mean | Sd | Sem | (% inhib) |
| PBS | 1 | 9.6 | 8.9 | 2.2 | 0.8 | |
| (s.c.) | 2 | 12.9 | | | | |
| | 3 | 7.2 | | | | |
| | 4 | 9.9 | | | | |
| | 5 | 6.6 | | | | |
| | 6 | 7.2 | | | | |
| | 7 | 10.5 | | | | |
| | 8 | 7.5 | | | | |
| H4 ANTAG #1 | 1 | 7.8 | 6.8 | 2.1 | 0.7 | 0.04 |
| (10 mg/kg; s.c.) | 2 | 4.5 | | | | (−24%) |
| | 3 | 3.0 | | | | |
| | 4 | 7.8 | | | | |
| | 5 | 8.1 | | | | |
| | 6 | 9.3 | | | | |
| | 7 | 6.6 | | | | |
| | 8 | 7.2 | | | | |

Mice were tested with either PBS (10 ml/kg) or H4 ANTAG #1 (10 mg/kg) at −15 minutes, +2 hours and +4 hours, and with 3 mg MSU crystals at Time 0. PMN influx into the peritoneal cavity was measured at the six-hour time point after collection of the lavage fluids and specific staining as described in the experimental section.

Conclusion

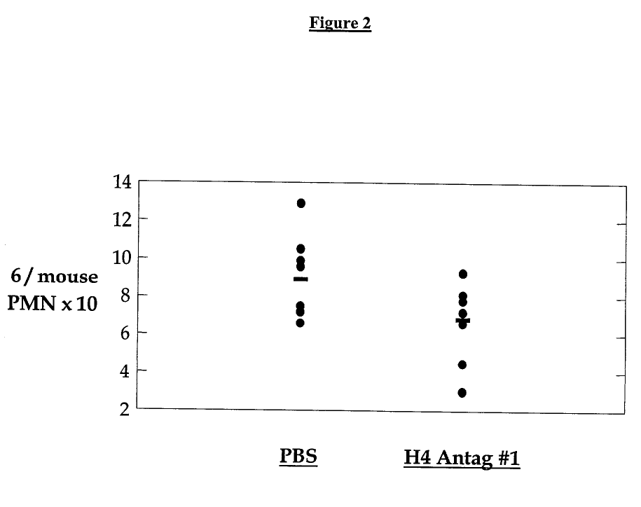

As expected, MSU crystals produced a PMN extravasation that was intense at the 6-hour time-point. Treatment with a specific histamine H4 receptor antagonist, H4 ANTAG #1, significantly reduced PMN migration (Table 2; FIG. 2): the degree of inhibition was 24%. To conclude, this study demonstrates that a histamine H4 receptor antagonist is effective in reducing PMN accumulation in an experimental model of cell recruitment in response to local application of MSU crystals in the mouse peritoneal cavity.

### EXAMPLE 3

### The Inhibition of Croton Oil Induced Topical Inflammation in Mice by Histamine H4 Receptor Antagonists

This example demonstrates the discovery for the first time that histamine H4 receptor antagonists can block the inflammation associated with topical application of croton oil.

Materials and Methods

Animals

Male or female ICR derived mice weighing 22±1 gms were used. Space allocation for five animals was 45×23×15 cm. Mice were housed in APEC R cages. All animals were maintained in a controlled temperature (22° C.-24° C.) and humidity (60% -80%) environment with twelve-hour light/dark cycles. Free access to standard lab chow for Mice (LabDiet Rodent Diet, PMI Nutrition International, USA) and tap water was granted.

Chemicals

Acetone (Wako, Japan), Croton oil (Sigma, USA), Indomethacin (Sigma, USA) and Pyrogen free saline (Astar, Taiwan).

Protocol Croton Oil Induced Topical Inflammation

Groups of five ICR derived male mice weighing 22±1 gms were used. H4 ANTAG #1 (10 mg/kg) and vehicle (0.9% NaCl) as well as the positive control Indomethacin (30 mg/kg) were administered subcutaneously to test animals at 30 minutes before and 2, 4 hours after Croton oil (8% in 20 μl acetone) was applied topically. Ear swelling was measured by Dyer model micrometer gauge six hours after Croton oil as an index of inflammation.

Results

TABLE 3
Effect of H4 ANTAG #1 on Croton Oil Induced Topical Inflammation

| | | Difference in ear | | | P value |
|---|---|---|---|---|---|
| Treatment | n | thickness (×0.01 mm) | Mean | Sem | (% inhib) |
| PBS | 1 | 12 | 16.6 | 1.4 | |
| (s.c.) | 2 | 17 | | | |
| | 3 | 15 | | | |
| | 4 | 19 | | | |
| | 5 | 20 | | | |
| H4 ANTAG #1 | 1 | 12 | 12.0 | 1.2 | 0.03 |
| (10 mg/kg; s.c.) | 2 | 10 | | | (−28%) |
| | 3 | 13 | | | |
| | 4 | 9 | | | |
| | 5 | 16 | | | |
| Indomethacin | 1 | 5 | 10.0 | 1.3 | 0.001 |
| (30 mg/kg; s.c.) | 2 | 10 | | | (−40%) |
| | 3 | 12 | | | |
| | 4 | 12 | | | |
| | 5 | 11 | | | |

Conclusions

In the croton oil induced topical inflammation ear swelling assay, a histamine H4 receptor antagonist, RJW 423640, at a dose of 10 mg/kg×3 (s.c.) significantly reduced the swelling with respect to the vehicle control. This effect was similar to Indomethacin (30 mg/kg×3). These results show that a histamine H4 receptor antagonist can act as an anti-inflammatory reagent.

### EXAMPLE 4

### Cloning of Human Histamine H4 Receptor cDNA into a Mammalian Expression Vector

The human histamine H4 receptor cDNAs (collectively referred to as pH4R) were cloned into the mammalian expression vector pCIneo. The human histamine H4 receptor cDNA clone was isolated from the human thalamus cDNA library. The full length cDNA was used as the template for PCR using specific primers with EcoR1 and Not1 sites for cloning. The PCR product was purified on a column (Wizard PCR DNA purification kit from Promega) and digested with Not I and EcoR1 (NEB) to create cohesive ends. The product was purified by a low melting agarose gel electrophoresis. The pCIneo vector was digested with EcoR1 and Not1 enzymes and subsequently purified on a low melt agarose gel. The linear vector was used to ligate to the human histamine H4 receptor cDNA inserts. Recombinants were isolated, designated human histamine H4 receptor, and used to transfect mammalian cells (SK-N-MC cells) by CaPO₄-DNA precipitation. Stable cell clones were selected by growth in the presence of G418. Single G418 resistant clones were isolated and shown to contain the intact human histamine H4 receptor gene. Clones containing the human histamine H4 receptor cDNAs were analyzed for pH4R expression by measuring inhibition of adenylate cyclase in response to histamine according to the method of (Konig et al., 1991) or by directly measuring cAMP accumulation by radioimmunoassay using Flashplates (NEN). Expression was also analyzed using [³H]-histamine binding assays (Clark et al., 1992). Recombinant plasmids containing human histamine H4 receptor encoding DNA were used to transform the mammalian COS7 or CHO cells or HEK293 or L-cells or SK-N-MC cells.

Cells expressing human histamine H4 receptor, stably or transiently, are used to test for expression of human histamine H4 receptor and for [³H]-histamine binding activity. These cells are used to identify and examine other compounds for their ability to modulate, inhibit or activate the human histamine H4 receptor and to compete for radioactive histamine binding.

Cassettes containing the human histamine H4 receptor cDNA in the positive orientation with respect to the promoter are ligated into appropriate restriction sites 3′ of the promoter and identified by restriction site mapping and/or sequencing. These cDNA expression vectors are introduced into fibroblastic host cells for example COS-7 (ATCC# CRL1651), and CV-1 tat [Sackevitz et al., Science (1987) 238:1575], 293, L (ATCC# CRL6362), SK-N-MC (ATCC# HTB-10) by standard methods including but not limited to electroporation, or chemical procedures (cationic liposomes, DEAE dextran, calcium phosphate). Transfected cells and cell culture supernatants are harvested and analyzed for human histamine H4 receptor expression as described herein.

All of the vectors used for mammalian transient expression can be used to establish stable cell lines expressing human histamine H4 receptor. Unaltered human histamine H4 receptor cDNA constructs cloned into expression vectors are expected to program host cells to make human histamine H4 receptor protein. The transfection host cells include, but are not limited to, CV-1-P [Sackevitz et al., Science (1987) 238:1575], tk-L [Wigler et al. Cell (1977) 11:223], NS/0, and dHFr-CHO [Kaufman and Sharp, J. Mol. Biol. (1982) 159:601].

Co-transfection of any vector containing human histamine H4 receptor cDNA with a drug selection plasmid including, but not limited to G418, aminoglycoside phosphotransferase; hygromycin, hygromycin-B phosphotransferase; APRT, xanthine-guanine phosphoribosyl-transferase, will allow for the selection of stably transfected clones. Levels of human histamine H4 receptor are quantitated by the assays described herein.

Human histamine H4 receptor cDNA constructs are also ligated into vectors containing amplifiable drug-resistance markers for the production of mammalian cell clones synthesizing the highest possible levels of human histamine H4 receptor. Following introduction of these constructs into cells, clones containing the plasmid are selected with the appropriate agent, and isolation of an over-expressing clone with a high copy number of plasmids is accomplished by selection in increasing doses of the agent.

The expression of recombinant human histamine H4 receptor is achieved by transfection of full-length human histamine H4 receptor cDNA into a mammalian host cell.

Characterization of Human Histamine H4 Receptor

Human SK-N-MC cells were transfected with pH4R and selected in the presence of neomycin for ten days. Individual colonies were picked and grown in six well-dishes. Cells were then plated onto 96 well-plates and grown to confluence. Cells were incubated for twenty minutes with isobutylmethylxanthine (1 mM). Cells were then stimulated with histamine (100 pM-100 uM) for five minutes. Cells were then stimulated with forskolin (3 uM) and allowed to incubate at 37° C. for twenty minutes. Cells were then treated with 0.1N hydrochloric acid. Cells were then frozen and thawed. Aliquots of the supernatant were then analyzed for their cyclic AMP content using a standard cAMP radioimmunoassay kit (Flashplates, NEN). The forskolin treatment raises the intracellular concentration of cAMP. Any cells that responded to histamine by decreasing the cAMP content in response to forskolin were considered to be expressing active functional human histamine H4 receptor. The recombinant human histamine H4 receptor expressed from the human histamine H4 receptor-encoding DNA molecule described herein was shown to be specifically activated by histamine.

### EXAMPLE 5

### Binding Assay on Recombinant Human Histamine H4 Receptor

SK-N-MC cells or COS7 cells that were transiently transfected with pH4R and grown in 150 cm² tissue culture dishes. Cells were washed with saline solution, scraped with a cell scraper and collected by centrifugation (1000 rpm, 5 min). SK-N-MC or COS7 cells expressing human histamine H4 receptor binds ³H-histamine with high affinity (FIG. 4). Cell membranes are prepared by homogenization of the cell pellet in 20 mM Tris-HCl with a polytron tissue homogenizer for ten seconds at high speed. Homogenate is centrifuged at 1000 rpm for five minutes at 4° C. The supernatant is then collected and centrifuged at 20,000×g for twenty-five minutes at 4° C. The final pellet is re-suspended in 50 mM Tris-HCl. Cell membranes are incubated with ³H-histamine (0.5 nM-70 nM) in the presence or absence of excess histamine (10000 nM). Incubation occurs at room temperature for forty-five minutes. Membranes are harvested by rapid filtration over Whatman GF/C filters and washed four times with ice cold 50 mM Tris HCl. Filters are then dried, mixed with scintillant and counted for radioactivity. SK-N-MC or COS7 cells expressing human histamine H4 receptor are used to measure the affinity of binding of other compounds and their ability to displace ³H-ligand binding by incubating the above described reaction in the presence of various concentrations of inhibitor or compound to be tested.

### EXAMPLE 6

### Ligand Binding to Mammalian Histamine H4 Receptors

The affinity of ³H-histamine for rat, mouse, guinea pig, and human histamine H4 receptors was determined using standard techniques as described herein. Saturation binding was performed on membranes from SK-N-MC cells stably transfected with the appropriate histamine H4 receptor. The Kd values were derived from a −1/slope of the linear regression of a Scatchard plot (bound/free vs. bound). The results are show in Table 4.

TABLE 4

| Species | ³H-histamine K_{d} (nM) |
|---|---|
| Rat | 105 |
| Murine | 34 |
| Guinea Pig | 20 |
| Human | 5 |

The relative affinity of several known histamine receptor ligands was determined by competitive binding of 30 nM ³H-histamine. Kᵢ values for each ligand were calculated according to the method of Cheng and Pruscoff (Kᵢ=IC₅₀/(1+[³H-histamine]/K_{d}). The Kd values for ³H-histamine were those set forth in Table 2. The results are presented in Table 5.

TABLE 5

| | Human Ki | Guinea Pig | Rat Ki | Murine |
|---|---|---|---|---|
| Compound | (nM) | Ki (nM) | (nM) | Ki (nM) |
| Imetit | 1.3 | 30 | 6.8 | 6.6 |
| Histamine | 5.9 | 27 | 70 | 41 |
| Clobenpropit | 4.9 | 3.6 | 63 | 14 |
| N-methylhistamine | 48 | 220 | 552 | 303 |
| Thioperamide | 52 | 83 | 28 | 22 |
| R-α-methylhistamine | 144 | 486 | 698 | 382 |
| Burimamide | 124 | 840 | 958 | 696 |
| Clozapine | 626 | 185 | 2200 | 2780 |

What is claimed is:

1. A method of identifying compounds that modulate mammalian histamine H4 receptor protein activity, comprising:
a) combining a putative modulator compound of mammalian histamine H4 receptor protein activity with mammalian histamine H4 receptor protein and a known histamine receptor H4 ligand; and
b) measuring an effect of the modulator on the protein function or its ability to bind the ligand, wherein said effect is inhibition, activation, antagonist, agonist or reverse agonist activity, wherein said modulator compound is a modulator of inflammation or inflammatory responses.

2. The method of claim 1, wherein the effect measured in step (b) is competition between the modulator of step (a) with a known ligand of the histamine H4 receptor for binding to the receptor.

3. The method of claim 1, wherein the effect measured in step (b) is modulation of a histamine H4 receptor intracellular second messenger.

4. The method of claim 3, wherein the intracellular second messenger is selected from a group consisting of cAMP, calcium, and a reporter gene product.

5. A compound identified using the method of claim 1, wherein said compound is an inhibitor of a mammalian histamine H4 receptor function and an inhibitor of inflammation or inflammatory responses in vivo or in vitro.

6. A compound identified using the method of claim 1, wherein said compound is an agonist, antagonist, or inverse agonist of a mammalian histamine H4 receptor and wherein said compound modulates inflammation or inflammatory responses in vitro or in vivo.

7. A compound identified using the method of claim 1, wherein said compound modulates the expression of the mammalian histamine H4 receptor protein and wherein said compound modulates inflammation or inflammatory responses in vitro or in vivo.

8. A pharmaceutical composition comprising a compound active in the method of claim 1 and a pharmaceutically acceptable carrier wherein said compound is a modulator of inflammation or inflammatory responses.

9. A method of treating a patient in need of such treatment to modulate inflammation or a disease or condition that is mediated by inflammation and histamine H4 receptor comprising administration of a pharmaceutical composition of claim 8.

10. A monospecific antibody immunologically reactive with a mammalian histamine H4 receptor protein, wherein said antibody modulates inflammation or inflammatory responses in vitro or in vivo.

11. The antibody of claim 10, wherein the antibody blocks histamine binding or activation of the mammalian histamine H4 receptor protein, wherein said antibody modulates inflammation or inflammatory responses in vitro or in vivo.

12. A method of identifying compounds that modulate mammalian histamine H4 receptor protein activity, comprising:
a) combining a putative modulator compound of mammalian histamine H4 receptor protein activity with mammalian histamine H4 receptor protein and a known histamine receptor H4 ligand; and
b) measuring an effect of the modulator on the protein function or its ability to bind the ligand, wherein said effect is inhibition, activation, antagonist, agonist or reverse agonist activity,
wherein said modulator compound is a modulator of polymorphonuclear leukocyte activation.

13. The method of claim 12, wherein the effect measured in step (b) is competition between the modulator of step (a) with a known ligand of the histamine H4 receptor for binding to the receptor.

14. The method of claim 12, wherein the effect measured in step (b) is modulation of a histamine H4 receptor intracellular second messenger.

15. The method of claim 14, wherein the intracellular second messenger is selected from a group consisting of cAMP, calcium, and a reporter gene product.

16. A compound identified using the method of claim 12, wherein said compound is an inhibitor of a mammalian histamine H4 receptor function and an inhibitor of polymorphonuclear leukocyte activation in vivo or in vitro.

17. A compound identified using the method of claim 12, wherein said compound is an agonist, antagonist, or inverse agonist of a mammalian histamine H4 receptor and wherein said compound modulates polymorphonuclear leukocyte activation in vitro or in vivo.

18. A compound identified using the method of claim 12, wherein said compound modulates the expression of the mammalian histamine H4 receptor protein and wherein said compound modulates polymorphonuclear leukocyte activation in vitro or in vivo.

19. A pharmaceutical composition comprising a compound active in the method of claim 12 and a pharmaceutically acceptable carrier wherein said compound is a modulator of polymorphonuclear leukocyte activation.

20. A method of treating a patient in need of such treatment to modulate inflammation or a disease or condition that is mediated by polymorphonuclear leukocyte activation and histamine H4 receptor comprising administration of a pharmaceutical composition of claim 19.

21. A monospecific antibody immunologically reactive with a mammalian histamine H4 receptor protein, wherein said antibody modulates polymorphonuclear leukocyte activation in vitro or in vivo.

22. The antibody of claim 21, wherein the antibody blocks histamine binding or activation of the mammalian histamine H4 receptor protein, wherein said antibody modulates polymorphonuclear leukocyte activation in vitro or in vivo.

23. A method of identifying compounds that modulate mammalian histamine H4 receptor protein activity, comprising:
a) combining a putative modulator compound of mammalian histamine H4 receptor protein activity with mammalian histamine H4 receptor protein and a known histamine receptor H4 ligand; and
b) measuring an effect of the modulator on the protein function or its ability to bind the ligand, wherein said effect is inhibition, activation, antagonist, agonist or reverse agonist activity,
wherein said modulator compound has is a modulator of mast cell activation.

24. The method of claim 23, wherein the effect measured in step (b) is competition between the modulator of step (a) with a known ligand of the histamine H4 receptor for binding to the receptor.

25. The method of claim 23, wherein the effect measured in step (b) is modulation of a histamine H4 receptor intracellular second messenger.

26. The method of claim 25, wherein the intracellular second messenger is selected from a group consisting of cAMP, calcium, and a reporter gene product.

27. A compound identified using the method of claim 23, wherein said compound is an inhibitor of a mammalian histamine H4 receptor function and an inhibitor of mast cell activation in vivo or in vitro.

28. A compound identified using the method of claim 23, wherein said compound is an agonist, antagonist, or inverse agonist of a mammalian histamine H4 receptor and wherein said compound modulates mast cell activation in vitro or in vivo.

29. A compound identified using the method of claim 23, wherein said compound modulates the expression of the mammalian histamine H4 receptor protein and wherein said compound modulates mast cell activation in vitro or in vivo.

30. A pharmaceutical composition comprising a compound active in the method of claim 23 and a pharmaceutically acceptable carrier wherein said compound is a modulator of mast cell activation.

31. A method of treating a patient in need of such treatment to modulate inflammation or a disease or condition that is mediated by mast cell activation and histamine H4 receptor comprising administration of a pharmaceutical composition of claim 30.

32. A monospecific antibody immunologically reactive with a mammalian histamine H4 receptor protein, wherein said antibody modulates mast cell activation in vitro or in vivo.

33. The antibody of claim 32, wherein the antibody blocks histamine binding or activation of the mammalian histamine H4 receptor protein, wherein said antibody modulates mast cell activation in vitro or in vivo.
